(19) United States
(12) Patent Application Publication
(10) Pub. No.: US 2008/0147120 A1
(43) Pub. Date: Jun. 19, 2008

(54) METAL INJECTION MOLDING OF SPINAL FIXATION SYSTEMS COMPONENTS

(76) Inventors: Fred Molz, Collierville, TN (US); Hai H. Trieu, Cordova, TN (US)

Correspondence Address: GOODWIN PROCTER / MEDTRONIC, 901 NEW YORK AVENUE , NW, WASHINGTON, DC, 20006, US

(21) Appl. No.: 12/037,731

(22) Filed: Feb. 26, 2008

Related U.S. Application Data
(63) Continuation of application No. 11/117,517, filed on Apr. 29, 2005.

(51) Int. Cl. A61B 17/58; B21D 53/00

(52) U.S. Cl. 606/246; 29/284

(57) ABSTRACT

A method of making a component of a spinal fixation system. The method may comprise providing a mixture of a powder of at least one metal or metal alloy and a polymeric binder. A metal-injection-molding process may use the mixture to form a component for a spinal fixation system. The components may have varying flexibility across their cross-sections. Also, components are provided that are produced by this process, such as spinal fixation rods and plates.

This application is a continuation of U.S. patent application Ser. No. 11/117,517, filed on Apr. 29, 2005, the disclosure of which is incorporated by reference herein in its entirety.

### FIELD OF THE INVENTION

Embodiments of the invention relate to methods for fabricating components for use in spinal fixation systems. More particularly, the embodiments relate to the use of metal-injection-molding to produce rods and plates having complex geometries for use in spinal fixation systems.

### BACKGROUND

The spinal (vertebral) column is a biomechanical structure composed primarily of ligaments, muscles, vertebrae, and intervertebral discs. The biomechanical functions of the spinal column include (i) support of the body; (ii) regulation of motion between the head, trunk, arms, pelvis, and legs; and (iii) protection of the spinal cord and the nerve roots. Damage to one or more components of the spinal column, such as an intervertebral disc, may result from disease or trauma and cause instability of the spinal column. To prevent further damage and overcome some of the symptoms resulting from a damaged spinal column, a spinal fixation device may be installed to stabilize the spinal column.

A spinal fixation device generally consists of stabilizing elements, such as rods or plates, attached by anchors to the vertebrae in the section of the vertebral column that is to be stabilized. The spinal fixation device restricts the movement of the fixed vertebrae relative to one another and supports at least a part of the stresses that would otherwise be imparted to the vertebral column. Typically, the stabilizing element is rigid and inflexible and is used in conjunction with an intervertebral fusion device to promote fusion between adjacent vertebral bodies. There are some disadvantages associated with the use of rigid spinal fixation devices, including decreased mobility, stress shielding (i.e. too little stress on some bones, leading to a decrease in bone density), and stress localization (i.e. too much stress on some bones, leading to fracture and other damage).

In response, flexible spinal fixation devices have been employed. These devices are designed to support at least a portion of the stresses imparted to the vertebral column but also allow a degree of movement. In this way, flexible spinal fixation devices avoid some of the disadvantages of rigid spinal fixation devices.

The description herein of problems and disadvantages of known apparatuses, methods, and devices is not intended to limit the invention to the exclusion of these known entities. Indeed, embodiments of the invention may include one or more of the known apparatus, methods, and devices without suffering from the disadvantages and problems noted herein.

### SUMMARY OF THE INVENTION

What is needed is an improved method to fabricate spinal fixation systems, and systems so fabricated that have a variable flexibility across their cross-section. Additionally, what is needed are components for spinal fixation systems that have complex external and internal geometries in order to provide for variable flexibility across their cross-section. Embodiments of the invention solve some or all of these needs, as well as additional needs.

Therefore, in accordance with an embodiment, there is provided a method of making a component of a spinal fixation system. The method comprises providing a mixture including a powder of at least one metal or metal alloy and a polymeric binder. The mixture is used in a metal-injection-molding process to form a spinal fixation system component. The component formed by the process may have a variable flexibility across its cross-section.

In accordance with another embodiment, there is provided a product comprising a spinal fixation system component made by a metal-injection-molding process as described herein.

These and other features and advantages of the embodiments will be apparent from the description provide herein.

### BRIEF DESCRIPTION OF THE DRAWINGS

FIG. 1, embodiments A-C, is a drawing of exemplary spinal fixation rods according to embodiments of the invention.

FIG. 2, embodiments A-D, is a drawing of exemplary spinal fixation rods according to embodiments of the invention.

FIG. 3, embodiments A-C, is a drawing of an exemplary spinal fixation plate according to embodiments of the invention.

### DETAILED DESCRIPTION OF THE EMBODIMENTS

The following description is intended to convey a thorough understanding of the various embodiments of the invention by providing a number of specific embodiments and details involving methods of fabricating components for use in spinal fixation systems and components produced thereby. It is understood, however, that the present invention is not limited to these specific embodiments and details, which are exemplary only. It is further understood that one possessing ordinary skill in the art, in light of known systems and methods, would appreciate the use of the invention for its intended purposes and benefits in any number of alternative embodiments.

An embodiment of the invention provides a method of making a component of a spinal fixation system. The method comprises providing a mixture including a powder of at least one metal or metal alloy and a polymeric binder. The spinal fixation system component may be formed from the mixture using a metal-injection-molding process. The component formed by the process may have a variable flexibility across its cross-section.

The components of the embodiments include, but are not limited to, rods, plates, screws, clamps, anchors, fusion cages, nucleus replacement devices, nails, and other components of spinal fixation systems. In preferred embodiments, there are provided rods and plates for spinal fixation systems that have varying flexibility across their cross-section. The varying flexibility of the rods and plates can be created in a number of different manners, some of which are described herein.

In a preferred embodiment, the components may be fabricated to be irregularly shaped. For example, in the case of a rod, the rod may be fabricated to have a varying external diameter. In portions or sections of the rod where more flexibility are desired, the rod may have a smaller diameter. In portions or sections of the rod where less flexibility is desired, the rod may have an increased diameter. In the case of a plate, the plate may be fabricated to have a varying thickness and/or width. In portions or sections of the plate where more flexibility are desired, the plate may be thinner and/or narrower. In portions or sections of the plate where less flexibility is desired, the plate may be thicker and/or wider.

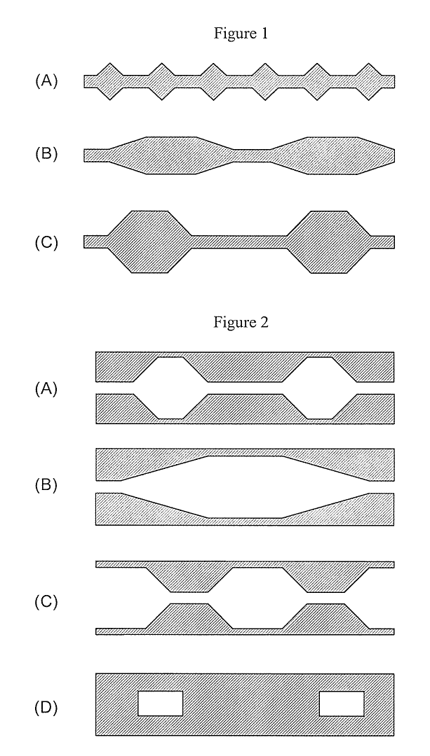

FIG. 1, embodiments A-C, illustrate some exemplary spinal fixation rods in accordance with the embodiments. As seen, the exemplary rods are irregularly shaped. In this case, the diameter varies along the length of the spinal fixation rod and across its cross-section. Portions or sections of the rods with increased diameter may be portions or sections of the rod where decreased flexibility is desired. Portions or sections of the rods with decreased diameter may be portions or sections of the rod where increased flexibility is desired.

In another preferred embodiment, the components may be fabricated to have one or more internal cavities. Internal cavities, for example, may be placed in sections or portions of the components where greater flexibility is desired. Internal cavities may be absent or smaller, preferably, in portions or sections of the components where less flexibility is desired. For example, larger cavities may be positioned in portions or sections of the components where greater flexibility is desired, and smaller cavities may be positioned in portions or sections of the components where less flexibility is desired. Additionally, the cavities may be irregularly shaped in order to vary the flexibility of the components. For example, a component may be hollow with an irregular internal shape so that the component may have varying flexibility along its cross-section.

FIG. 2, embodiments A-D, illustrates spinal fixation rods having internal cavities in accordance with embodiments of the invention. In embodiments A, B, and C, the rods all have a regular external shape but an irregularly shaped longitudinal cavity within the interior of the rods. As a result of the cavities, the rods have an irregular internal shape such that the sidewall thickness of the rods varies along its longitudinal extent. Generally, portions or sections of the rods with thicker sidewalls correspond to portions or sections of the rods where less flexibility is desired, and portions or sections of the rods with thinner sidewalls correspond with portions or section of the rod were more flexibility is desired. In embodiment D, an irregularly shaped rod is illustrated having two regularly shaped internal cavities. Selective placement of internal cavities within the rods and plates of the embodiments may allow selected portions or sections of the rods and plates to be more flexible than are other portions or sections of the rods and plates.

In embodiments where the components have internal cavities, a polymeric material may be located within the cavities. For example, a polymeric material may be desirable in the cavities in order to impart increased stiffness or strength to the components compared to components with un-filled internal cavities. The polymeric material may be placed in the cavities, for example, by injection molding the material into the cavities following fabrication of the components. For example, the cavities in the rods illustrated in FIGS. 1 and 2 may be filled, partially or fully, with one or more polymeric materials. The polymeric material may be delivered to the cavities by injecting the polymers therein, or in another applicable fashion in accordance with the guidelines herein.

Any applicable polymeric material may be located within the cavities. For example, a resorbable polymeric material such as polylactides (PLA), polyglycolide (PGA), copolymers of (PLA and PGA), polyorthoesters, tyrosine, polycarbonates, and mixtures and combinations thereof may be placed in the cavities. Alternatively, a non-resorbable polymeric material such as a member of the polyaryletherketone family, polyurethanes, silicone polyurethanes, polyimides, polyetherimides, polysulfones, polyethersulfones, polyaramids, polyphenylene sulfides, and mixtures and combinations thereof may be placed in the cavities.

In another embodiment, the components may have irregular cross-sectional geometries. For example, the components may have different cross-sectional geometries in different portions or sections of the components. The cross-sectional geometries may be, but are not limited to, circular, elliptical, square, rectangular, triangular, pentagonal, hexagonal, heptagonal, octagonal, irregular, and so forth. Therefore, the cross-sectional geometry of the components may change, for example, from one geometry to another along its longitudinal extent in the case of a spinal fixation rod or plate.

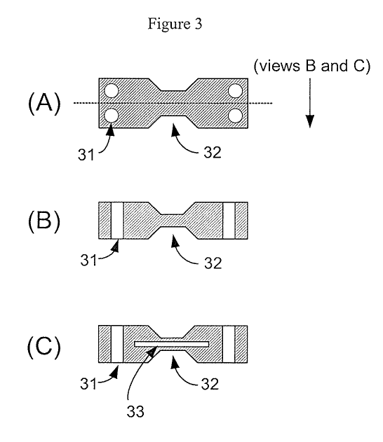

FIG. 3, embodiments A-C, illustrates a spinal fixation plate in accordance with the embodiments. As seen in embodiment A (plane view), the spinal fixation plate is irregularly shaped. In this case, the width of the plate changes along its longitudinal length; the mid-section 32 of the plate is narrower than at its ends. Therefore, all other things being equal, the plate will be more flexible in its mid-section 32 than at its ends. Apertures 31 are provided in order to affix the plate to adjacent vertebrae using, for example, bone screws. In embodiment B, a cross-section of the plate is shown. The cross sectional shape of the plate also is irregular; the mid-section 32 of the plate is thinner than the ends. Again, the thinner mid-section may be more flexible than the thicker areas at the ends of the plate. In embodiment C, a cross-section of the plate is shown wherein the plate further comprises an internal cavity 33 positioned at its mid-section 32. The internal cavity may further extenuate the differences in flexibility between the mid-section of the plate and its ends. Optionally, the internal cavity may be partially or fully filled with a polymeric material in order to adjust the flexibility of the plate.

It should be apparent that the components provided by the embodiments may take a myriad of different forms or configurations, in accordance with the guidelines provided herein. Therefore, one of skill in the art will appreciate still other configurations for spinal fixation components in accordance with the embodiments. It will be appreciated, for example, that an infinite number of variations in cross sections of the metal-injection-molded rods and plates provided by the embodiments may occur, in accordance with the guidelines provided herein.

Metallic components having complex internal and external shapes may be produced using metal-injection-molding (“MIM”) processes. MIM and feedstocks for use therein have been described, for example, in U.S. Pat. Nos. 4,694,881, 4,694,882, 5,040,589, 5,064,463, 5,577,546, 5,848,350, 6,860,316, 6,838,046, 6,790,252, 6,669,898, 6,619,370, 6,478,842, 6,470,956, 6,350,328, 6,298,901, 5,993,507, 5,989,493, the disclosures of each of which are incorporated herein in their entireties.

MIM facilitates the production of complex shaped components containing metal and metal alloys. For example, metal and metal alloys such as titanium, titanium alloys, tantalum, tantalum alloys, stainless steel alloys, cobalt-based alloys, cobalt-chromium alloys, cobalt-chromium-molybdenum alloys, niobium alloys, zirconium alloys, nickel, nickel alloys, and mixtures thereof may be used to fabricate components according to embodiments of the invention. MIM advantageously allows complex shaped metallic components to be produced at a much lower cost than by forging and casting processes. Therefore, MIM is highly advantageous for the production of components in spinal fixation systems that are irregularly shaped, such as the plates and rods described herein.

In general, the MIM process involves mixing a powder metal or metal alloy and a binder. Preferably, the mixture comprises a binder that is an organic aqueous based gel, and the mixture further comprises water. The mixed powder metal and binder composition preferably produces a generally flowable thixotropic mixture at relatively low temperature and pressure. The proportion of binder to powder metal may be about 40-60% binder by volume. Preferably, a flowable mixture with a viscosity is produced such that the mixture will fill all of the crevices and small dimensional features of a mold. The flowable mixture typically may be transferred to the mold via an injection molding machine.

Injection molding machines are known in the art and typically are capable of applying several hundred tons of pressure to a mold. The mold may be constructed with internal cooling passages to solidify the flowable material prior to removal. The mold cavity typically is larger than that of the desired finished part to account for the shrinkage that may occur after binder removal. The mold structure may be formed from either a rigid or a flexible material, such as metal, plastic, or rubber. Preferably, the mold is equipped with vents or bleeder lines to allow air to escape from the mold during the molding process. Alternatively, the mold may be equipped with a porous metal or ceramic insert to allow air to escape from the mold. After the mold has been filled with the flowable mixture, pressure may be applied to the mold/mixture to form the molded part, otherwise known as the preform. Typical injection mold pressures for a preform are in the range of about 10-12 ksi. The molded preforms may be referred to as “green” parts. The green preform may be dried by oven heating to a temperature sufficient to vaporize most of the remaining water. Then, the preform may be placed in a furnace to vaporize the binder. To achieve a part with high density and thus a sufficient working strength, the preform subsequently may be sintered.

Sintering is an elevated temperature process whereby a powder metal preform may be caused to coalesce into an essentially solid form having the same or nearly the same mechanical properties as the material in cast or wrought form. Generally, sintering refers to raising the temperature of the powder metal preform to a temperature close to, but not exceeding, the melting point of the material, and holding it there for a defined period of time. Under these conditions, interparticulate melting occurs and the material densifies to become solid.

In the case of MIM processes, the sintering process preferably causes interparticulate melting within the metallic component of the part while at the same time removing the binder component, which melts and vaporizes at a much lower temperature than does the metallic component. The resulting structure may be a high-density metallic piece substantially or completely free of the binder component. MIM molding facilitates the production of smaller and more dimensionally complex metallic pieces than does typical forging or casting processes because of the flexibility of the injection molding step in the process. One skilled in the art will appreciate the modifications of the basic MIM process that may be used in the embodiments, in accordance with the guidelines herein.

Described herein are some exemplary spinal fixation systems utilizing rods, plates, and other components. It is contemplated that the metal-injection-molded components of the embodiments can be substituted for the rods, plates, and other components of these exemplary spinal fixation systems. In a preferred embodiment, components according to the embodiments are molded to replicate the external geometries of these rods and plates, but with internal cavities to create portions or sections of the rods and plates with varying flexibility. The metal-injection-molded components may be used to, for example, repair or customize the following spinal fixation systems.

U.S. Pat. No. 6,858,029 discloses a system for fixing vertebrae comprising clamps and a connection portion to which the clamps may be mounted. The clamps are designed to engage vertebral bodies and the connection portion may comprise a rod. The system components disclosed in the '029 patent (e.g., rods, screws, etc.) may be fabricated using the MIM process described in the embodiments herein. The disclosure of U.S. Pat. No. 6,858,029 is incorporated herein by reference in its entirety.

U.S. Pat. No. 6,843,790 discloses a system for rigidly coupling at least three vertebrae. The system comprises an elongated plate having an upper and a lower surface, a first upper linear section, a second lower linear section, and a central curved section. The lower linear section and upper linear sections may be at an angle relative to each other. An opening is located within the central region of the plate and runs along the central axis of the plate. The plate may be affixed to the vertebrae by a plurality of bone engaging screws, each having a head for engaging the aperture in the plate. Components of the system disclosed in the '790 patent, including the elongated plate, may be fabricated using the MIM process described in the embodiments herein. The disclosure of U.S. Pat. No. 6,843,790 is incorporated herein by reference in its entirety.

U.S. Pat. No. 6,770,075 discloses a spinal fixation system including a plurality of anchor screw assemblies having anchor screws and clamp assemblies defining rod passages therethrough. A rod is receivable in the rod passages between the anchor screw assemblies, and a spacer is securable on the rod. Anchor screw assemblies can be affixed to adjacent vertebrae and the rod can be secured between the anchor screw assemblies, thereby fixing a relative spacing of the adjacent vertebrae. Components of the system disclosed in the '075 patent, including the fixation rods, may be fabricated using the MIM process described in the embodiments herein. The disclosure of U.S. Pat. No. 6,770,075 is incorporated herein by reference in its entirety.

U.S. Pat. No. 6,740,088 discloses a spinal fixation system comprising a plate having curvature in two planes such that it conforms to the curvature of the L5 vertebral body and to the patient's lordotic curve. The plate has holes for receiving screws to anchor the plate to the vertebral body and sacrum. The plate's base has a flange or foot portion to provide a wider base end area for support in the L5-S1 vertebral interspace. The foot portion also is arranged for appropriate entry angle of screws into the sacrum such as to improve anchorage in the sacrum. Components of the system disclosed in the '088 patent, including the curved plate, may be fabricated using the MIM molding process described in the embodiments herein. The disclosure of U.S. Pat. No. 6,740,088 is incorporated herein by reference in its entirety.

U.S. Pat. No. 6,706,044 discloses a spinal fixation system consisting of at least two bone anchors for attaching the device to the spine, at least two stacked rods running generally parallel to one another, means for connecting the rods to the bone anchors, and means for compressing the rods tightly together. The at least two stacked rods have a longitudinal shape and length, a cross sectional shape and cross sectional diameter, and are immediately adjacent one another along their length. Components of the system disclosed in the '044 patent, including the stacked rods, may be fabricated using the MIM molding process described in the embodiments herein. The disclosure of U.S. Pat. No. 6,706,044 is incorporated herein by reference in its entirety.

U.S. Pat. No. 6,613,051 discloses a spinal fixation system comprising a support member defining a plurality of engaging portions thereon. At least two of the engaging portions are spaced longitudinally from each other and are adapted to span at least one vertebra. At least two of the engaging portions are spaced laterally from each other and adapted to span a lateral distance of the vertebra. A plurality of fixation elements are provided to mount the engaging portions onto the vertebra. The support member thereby is restrained from rotational or translational movement relative to the vertebra. Components of the system disclosed in the '051 patent, including the engaging portions, may be fabricated using the MIM molding process described in the embodiments herein. The disclosure of U.S. Pat. No. 6,613,051 is incorporated herein by reference in its entirety.

U.S. Pat. No. 6,599,290 discloses a spinal fixation system comprising a plate member having multiple pairs of nodes. Each node defines a bone screw aperture. Linking segments connect the pairs of nodes to one another and elongated viewing windows are located between adjacent linking segments. Components of the system disclosed in the '290 patent, including the plate member, may be fabricated using the MIM molding process described in the embodiments herein. The disclosure of U.S. Pat. No. 6,599,290 is incorporated herein by reference in its entirety.

U.S. Pat. No. 6,547,790 discloses a bone plate that is T-shaped and includes two apertures, one on each arm of the T, to accommodate bolt anchor assemblies to which a linking member (e.g., a rod or cable) may be attached. Three chamfered holes extend along the midline of the bone plate for bone screws, and one additional bone screw opening is provided on each side aim of the bone plate to firmly fasten the plate. The arms of the plate may curve, or extend at a slight dihedral angle to the central line of the T to conform to the skull. Components of the system disclosed in the '790 patent, including the T-shaped bone plate, may be fabricated using the MIM molding process described in the embodiments herein. The disclosure of U.S. Pat. No. 6,547,790 is incorporated herein by reference in its entirety.

The spinal fixation systems, including rods and plates described herein, are exemplary only and it is to be understood that the rods and plates provided by embodiments of the invention can be fabricated to be physically similar in external appearance and dimensions to any known system, rod, plate, or other component useful for spinal fixation. Therefore, the components of the embodiments generally can be substituted for one or more components of any given spinal fixation system. The components are not limited to a certain form or dimensions.

In a preferred embodiment, the spinal fixation rods and plates provided by the embodiments are fabricated using MIM to produce replacement rods and plates for use with known spinal fixation systems. Preferably, the replacement rods and plates are shaped to be compatible with known spinal fixation systems. In other words, the replacement rods and plates may have an external geometry, size, shape, dimensions, and so forth for use with known spinal fixation systems. In a more preferred embodiment, the replacement rods and plates may have one or more internal cavities therein. The internal cavities may be regularly or irregularly shaped and may be partially or fully filled with a polymeric material. In this way, the replacement rods and plates may be made to have a varying flexibility across their cross-sections. Therefore, the embodiments provide for replacement rods and plates for use with spinal fixation systems wherein the replacement rods and plates have varying flexibility across their cross-sections.

The components of the spinal fixation systems produced by MIM may have a much higher degree of dimensional and geometrical complexity than components produced using typical forging and casting processes. A more complex shape may be advantageous because complex shapes, such as those in FIGS. 1, 2, and 3, may result in components having variable flexibility across their cross-sections. Variable flexibility may be an advantageous property for a spinal fixation system component, particularly a rod or plate, in order to minimize stress shielding and other detrimental effects of using substantially rigid spinal fixation systems.

The foregoing detailed description is provided to describe the invention in detail, and is not intended to limit the invention. Those skilled in the art will appreciate that various modifications may be made to the invention without departing significantly from the spirit and scope thereof.

What is claimed is:

1. A method of making a component of a spinal fixation system, comprising:
providing a mixture comprising a powder of at least one metal or metal alloy and a polymeric binder; and
forming the component from the mixture using a metal-injection- molding process;
wherein the component has a variable flexibility across its cross-section.

2. The method of claim 1, wherein the powder of at least one metal or metal alloy is selected from the group of metals and metal alloys consisting of titanium, titanium alloys, tantalum, tantalum alloys, stainless steel alloys, cobalt-based alloys, cobalt-chromium alloys, cobalt-chromium-molybdenum alloys, niobium alloys, zirconium alloys, nickel, nickel alloys, and mixtures thereof.

3. The method of claim 1, wherein the component is irregularly shaped.

4. The method of claim 1, wherein the cross-section of the component is irregularly shaped.

5. The method of claim 1, wherein the component has at least one interior cavity.

6. The method of claim 5, wherein the interior cavity is irregularly shaped.

7. The method of claim 5, wherein the interior cavity contains a polymeric material.

8. The method of claim 7, wherein the polymeric material is a resorbable polymeric material selected from the group consisting of polylactides (PLA), polyglycolide (PGA), copolymers of (PLA and PGA), polyorthoesters, tyrosine, polycarbonates, and mixtures and combinations thereof.

9. The method of claim 7, wherein the polymeric material is a non-resorbable polymeric material selected from the group consisting of members of the polyaryletherketone family, polyurethanes, silicone polyurethanes, polyimides, polyetherimides, polysulfones, polyethersulfones, polyaramids, polyphenylene sulfides, and mixtures and combinations thereof.

10. A component having variable flexibility across its cross-section comprised of at least one metal or metal alloy and a polymer for use with a spinal fixation system, prepared by
providing a mixture comprising a powder of at least one metal or metal alloy and a polymeric binder; and
forming the component from the mixture using a metal-injection-molding process,
wherein the polymer is selected from resorbable and non-resorbable polymeric materials selected from the group consisting of polylactides (PLA), polyglycolide (PGA), copolymers of (PLA and PGA), polyorthoesters, tyrosine, polycarbonates, members of the polyaryletherketone family, polyurethanes, silicone polyurethanes, polyimides, polyetherimides, polysulfones, polyethersulfones, polyaramids, polyphenylene sulfides, and mixtures and combinations thereof.

11. The component of claim 10, wherein the metal or metal alloy is selected from the group consisting of titanium, titanium alloys, tantalum, tantalum alloys, stainless steel alloys, cobalt-based alloys, cobalt-chromium alloys, cobalt-chromium-molybdenum alloys, niobium alloys, zirconium alloys, nickel, nickel alloys, and mixtures thereof.

12. The component of claim 10, wherein the component is irregularly shaped.

13. The component of claim 10, wherein the cross-section of the component is irregularly shaped.

14. The component of claim 10, wherein the component has at least one interior cavity.

15. The component of claim 14, wherein the interior cavity is irregularly shaped.

16. The component of claim 14, wherein the interior cavity contains the polymeric material.

17. The component of claim 10, wherein the polymeric material is a resorbable polymeric material selected from the group consisting of polylactides (PLA), polyglycolide (PGA), copolymers of (PLA and PGA), polyorthoesters, tyrosine, polycarbonates, and mixtures and combinations thereof.

18. The component of claim 10, wherein the polymeric material is a non-resorbable polymeric material selected from the group consisting of members of the polyaryletherketone family, polyurethanes, silicone polyurethanes, polyimides, polyetherimides, polysulfones, polyethersulfones, polyaramids, polyphenylene sulfides, and mixtures and combinations thereof.

19. The component of claim 10, wherein the component is a rod.

20. The component of claim 10, wherein the component is a plate.
